(19) United States
(12) Patent Application Publication
(10) Pub. No.: US 2026/0114762 A1
(43) Pub. Date: Apr. 30, 2026

(54) METHOD AND APPARATUS FOR CALIBRATION TO REDUCE COUPLING BETWEEN SIGNALS IN A MEASUREMENT SYSTEM

(71) Applicant: Masimo Corporation, Irvine, CA (US)

(72) Inventors: Walter M. Weber, Laguna Hills, CA (US); Ammar Al-Ali, San Juan Capistrano, CA (US); Mohamed K. Diab, Ladera Ranch, CA (US); Marcelo M. Lamego, Cupertino, CA (US)

(21) Appl. No.: 18/943,755

(22) Filed: Nov. 11, 2024

Related U.S. Application Data
(63) Continuation of application No. 18/309,187, filed on Apr. 28, 2023, now Pat. No. 12,171,552.
Application No. 18/309,187 is a continuation of application No. 16/993,101, filed on Aug. 13, 2020, now Pat. No. 11,672,447.
Application No. 16/993,101 is a continuation of application No. 15/834,965, filed on Dec. 7, 2017, now Pat. No. 10,772,542.
Application No. 15/834,965 is a continuation of application No. 14/836,635, filed on Aug. 26, 2015, now Pat. No. 9,861,305.
Application No. 14/836,635 is a continuation of application No. 13/526,376, filed on Jun. 18, 2012, now abandoned.
Application No. 13/526,376 is a continuation of application No. 11/871,636, filed on Oct. 12, 2007, now abandoned.
(60) Provisional application No. 60/851,112, filed on Oct. 12, 2006.

(51) Int. Cl. A61B 5/1455; A61B 5/00; A61B 5/1495

(52) U.S. Cl. CPC A61B 5/1455; A61B 5/1495; A61B 5/7228; A61B 2562/0238

(57) ABSTRACT

A method and an apparatus for separating a composite signal into a plurality of signals is described. A signal processor receives a composite signal and separates a composite signal into separate output signals. Feedback from one or more of the output signals is provided to a configuration module that configures the signal processor to improve a quality of the output signals. In one embodiment, calibration data from multiple calibration data sets is used to configure the demodulation of the composite signal into separate output signals.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

The present application is a continuation of U.S. application Ser. No. 18/309,187, filed Apr. 28, 2023, titled “METHOD AND APPARATUS FOR CALIBRATION TO REDUCE COUPLING BETWEEN SIGNALS IN A MEASUREMENT SYSTEM”, which is a continuation of U.S. application Ser. No. 16/993,101, filed Aug. 13, 2020, titled “METHOD AND APPARATUS FOR CALIBRATION TO REDUCE COUPLING BETWEEN SIGNALS IN A MEASUREMENT SYSTEM”, now U.S. Pat. No. 11,672,447, which is a continuation of U.S. application Ser. No. 15/834,965, filed Dec. 7, 2017, titled “METHOD AND APPARATUS FOR CALIBRATION TO REDUCE COUPLING BETWEEN SIGNALS IN A MEASUREMENT SYSTEM”, now U.S. Pat. No. 10,772,542, which a continuation of U.S. application Ser. No. 14/836,635, filed Aug. 26, 2015, titled “METHOD AND APPARATUS FOR CALIBRATION TO REDUCE COUPLING BETWEEN SIGNALS IN A MEASUREMENT SYSTEM”, now U.S. Pat. No. 9,861,305, which is a continuation of U.S. application Ser. No. 13/526,376, filed Jun. 18, 2012, titled “METHOD AND APPARATUS FOR CALIBRATION TO REDUCE COUPLING BETWEEN SIGNALS IN A MEASUREMENT SYSTEM”, which is a continuation of U.S. patent application Ser. No. 11/871,636, filed Oct. 12, 2007, titled “METHOD AND APPARATUS FOR CALIBRATION TO REDUCE COUPLING BETWEEN SIGNALS IN A MEASUREMENT SYSTEM”, which claims priority benefit of U.S. Provisional Application No. 60/851,112, filed Oct. 12, 2006, titled “METHOD AND APPARATUS FOR CALIBRATION TO REDUCE COUPLING BETWEEN SIGNALS IN A MEASUREMENT SYSTEM”, the entire contents of which is hereby incorporated by reference.

### BACKGROUND

### Field of the Disclosure

The present invention relates to the field of signal processing, and more particularly, relates to multi-channel demodulators for demodulating mixed signals, such as, for example, signals generated in a pulse oximetry system.

### Background

In many multi-channel measurement and communication systems, crosstalk between channels and corruption of data within the channels are significant problems. Such problems can arise from variations in manufacturing tolerances, movement, propagation delays, phase shifts, temperature effects, degradation of components due to age or other factors, noise, etc.

A pulse oximetry system is one example of a system where the above-referenced problems are found. In a pulse oximetry system, blood oxygen saturation is determined by transmitting pulses of electromagnetic energy through a portion of a subject having blood flowing therein (e.g., through a finger, through an ear lobe, or other portion of the body where blood flows close to the skin). The pulses of electromagnetic energy comprise periodic pulses of red light having wavelengths of approximately 660 nanometers, for example, and periodic pulses of infrared light having wavelengths of approximately 905 nanometers.

After propagating through the portion of the subject, the red pulses and the infrared pulses are detected by a detector which is responsive to light at both wavelengths and which generates an electrical signal that has a relationship to the intensity of the electromagnetic energy incident on the detector. The detector output is a two-channel signal having a first signal component corresponding to the detected red pulses and a second signal component corresponding to the detected infrared pulses.

The two-channel signal is demodulated to recover separate signals corresponding to the first signal component and the second signal component. However, prior art demodulators are not sufficiently accurate enough to completely separate the two signal components in all cases. Thus, it is not uncommon for the first demodulator output signal (corresponding to the first signal component) to contain residual components of the second signal and vice versa. This crosstalk between the first and second signal components reduces the accuracy of the recovered first and second signals. In multi-channel systems with more than two channels, crosstalk can occur between all of the channels, again reducing accuracy.

### SUMMARY

The present invention solves these and other problems by separating a combined multi-channel signal into a plurality of output signals in a manner that reduces crosstalk and other contamination in the plurality of output signals. In one embodiment, the separator includes a multi-channel demodulator that is first configured using nominal values for the various components in the signal path. In one embodiment, the multi-channel demodulator is further configured using data obtained from calibration measurements. In one embodiment, the calibration measurements are made during an initialization period. In one embodiment, the calibration measurements are made frequently, continuously, or at selected intervals. In one embodiment, calibrations are performed on at least one of, initialization, on command, on attachment of a new sensor, continuously, and/or interspersed with measurements. In one embodiment of a system for measuring one or more blood constituents, the calibration measurements are made when the system detects that a patient has been connected to the system. In one embodiment, the multi-channel demodulator is further configured at regular intervals by re-running the calibration measurements. In one embodiment, the multi-channel demodulator comprises an optimizing demodulator. In one embodiment, crosstalk in the multi-channel demodulator is reduced by computing an amplitude and/or phase adjustment of one or more demodulation signals that are provided respectively to one or more mixers.

In one embodiment, an apparatus for measuring blood oxygenation in a subject includes a first signal source which applies a first input signal during a first time interval. A second signal source applies a second input signal during a second time interval. A detector detects a first parametric signal responsive to the first input signal passing through a portion of the subject having blood therein and detects a second parametric signal responsive to the second input signal passing through the portion of the subject. The detector generates a detector output signal responsive to the first and second parametric signals. A signal processor receives the detector output signal and demodulates the detector output signal by applying a first demodulation signal to a signal responsive to the detector output signal to generate a first demodulator output signal and applying a second demodulation signal to the signal responsive to the detector output signal to generate a second demodulator output signal. In one embodiment, the first demodulation signal has at least one component comprising a first frequency, a first phase, and a first amplitude; and the second demodulation signal has at least one component comprising a second frequency, a second phase, and a second amplitude. In one embodiment, the first phase and the second phase are chosen to reduce crosstalk from the first parametric signal to the second demodulator output signal and to reduce crosstalk from the second parametric signal to the first demodulator output signal. In one embodiment, the first amplitude and the second amplitude are chosen to reduce crosstalk from the first parametric signal to the second demodulator output signal and to reduce crosstalk from the second parametric signal to the first demodulator output signal. In one embodiment, at least one of the first amplitude, the first phase, the second amplitude, and the second phase are chosen to reduce crosstalk from the first parametric signal to the second demodulator output signal and to reduce crosstalk from the second parametric signal to the first demodulator output signal.

In one embodiment, at least one of the first amplitude, the first phase, the second amplitude, and the second phase is determined by turning off one of the first and second signal sources and measuring the crosstalk between one of the parametric signals and the non-corresponding output signal.

One embodiment includes a method of reducing crosstalk between two signals generated by applying a first pulse and a second pulse to measure a parameter. The first pulse and the second pulse are applied periodically at a repetition rate defining a period. The first pulse is generated during a first interval in each period and the second pulse is generated during a second interval in each period. In one embodiment, the second interval is spaced apart from the first interval. In one embodiment, the second interval overlaps at least a portion of the first interval. The first and second pulses produce first and second parametric signals responsive to the parameter. The first and second parametric signals are received by a detector which outputs a composite signal responsive to the first and second parametric signals. The method includes applying a first demodulation signal to the composite signal to generate a first demodulated output signal. The first demodulation signal includes at least one component having at least a first amplitude and a first phase. The method further includes applying a second demodulation signal to the composite signal to generate a second demodulated output signal. The second demodulation signal includes at least one component having at least a second amplitude and a second phase. The method further includes lowpass filtering the first demodulated output signal to generate a first recovered output signal responsive to the first parametric signal, and lowpass filtering the second demodulated output signal to generate a second recovered output signal responsive to the second parametric signal. The method also includes choosing at least one of the first phase, the first amplitude, the second phase, and the second amplitude to reduce crosstalk components in the first recovered output signal and the second recovered output signal. In one embodiment, the method also includes choosing the first phase and/or the second phase to reduce crosstalk components in the first recovered output signal and the second recovered output signal.

In one embodiment, the first phase and the second phase are chosen by applying a first light pulse pattern (activation pattern) during a first time period and measuring the first recovered output during the first time period as a first calibration output, and measuring the second recovered output during the first time period as a second calibration output. The method includes applying a second light pulse activation pattern during a second time period and measuring the first recovered output during the first time period as a third calibration output and measuring the second recovered output during the second time period as a fourth calibration output. The method further includes computing the first phase and the second phase from at least the first calibration output, the second calibration output, the third calibration output, and the fourth calibration output.

In one embodiment, the first phase is computed from a ratio of the first calibration output and the second calibration output.

In one embodiment, the first demodulation signal includes a sum of a first demodulation component having a first amplitude and a second demodulation component having a second amplitude. The second demodulation component is in quadrature with the first demodulation component and the act of choosing the first phase involves choosing the first amplitude and the second amplitude. In one embodiment, the quadrature components are sinusoidal and cosinusoidal.

In one embodiment, the first demodulation signal includes a sum of a sinusoidal component having a first amplitude and a cosinusoidal component having a second amplitude. The first amplitude and the second amplitude are chosen by a least squares minimization of an error corresponding to the crosstalk. In one embodiment, the error is integrated over a time period corresponding to an integer number of cycles of the sinusoidal component.

In one embodiment, a first demodulation signal is applied to a composite signal having first and second coefficients to generate a first demodulated signal. The first demodulation signal includes a first component having a first amplitude and a second component having a second amplitude. The first and second components being in quadrature. The second amplitude has a predetermined relationship to the first amplitude. The predetermined relationship is selected to cause the first demodulated signal to have lower frequency components that include a primary component corresponding primarily to the first desired component and a residual component corresponding to the second component. The first demodulated signal is lowpass filtered to generate a first output signal. At least one of the first amplitude and the second amplitude are adjusted to reduce the residual component with respect to the primary component.

In one embodiment, a pulse oximetry system includes a modulation signal generator. The modulation signal generator generates a first modulation signal including a first pulse at a repetition frequency having a first duty cycle. The modulation signal generator generates a second modulation signal including a second pulse which also repeats at the repetition frequency and having a second duty cycle. The second pulse can be non-overlapping with respect to the first pulse, or the second pulse can partially or completely overlap the first pulse. The first and second pulses include a plurality of components wherein a first component has a frequency corresponding to the repetition frequency and a second component has a second frequency corresponding to twice the first frequency. A first transmitter emits electromagnetic energy at a first wavelength in response to the first pulse. A second transmitter emits electromagnetic energy at a second wavelength in response to the second pulse. A detector receives electromagnetic energy at the first and second wavelengths after passing through a portion of a subject. The detector generates a detector output signal responsive to the received electromagnetic energy. The detector output signal includes a signal component responsive to attenuation of the electromagnetic energy at the first wavelength and a signal component responsive to attenuation of the electromagnetic energy at the second wavelength. A first demodulator multiplies the detector signal by a first demodulation signal and generates a first demodulated output signal. A second demodulator multiplies the detector signal by a second demodulation signal and generates a second demodulated output signal. A configuration module configures the first demodulation signal and the second demodulation signal to substantially separate the first demodulator output and the second demodulator output.

In one embodiment, the configuration module selects a phase relationship between the first demodulation signal and the second demodulation signal.

In one embodiment, the configuration module configures the first demodulation signal and the second demodulation signal using, at least in part, data obtained during a calibration period. In one embodiment, the calibration data includes first and second calibration data corresponding to the first and second demodulated output signals during a first time period, and third and fourth calibration data corresponding to the first and second demodulated output signals during a second time period. In one embodiment, the second transmitter is turned off during the first time period, and the first transmitter is turned off during the second time period.

In one embodiment, the configuration module configures the first demodulation signal and the second demodulation signal by adjusting initial parameters that define the first demodulation signal and the second demodulation signal. The configuration module adjusts the initial parameters using, at least in part, the calibration data obtained during a calibration period.

One embodiment includes an apparatus for measuring one or more blood constituents in a subject, wherein a first signal source applies a first input signal during a first time interval, a second signal source applies a second input signal during a second time interval, and a third signal source applies a second input signal during a third time interval. A detector detects a first parametric signal responsive to the first input signal passing through a portion of the subject having blood therein, a second parametric signal responsive to the second input signal passing through the portion of the subject, and a third parametric signal responsive to the third input signal passing through a portion of the subject having blood therein. The detector generates a detector output signal responsive to the first, second, and third parametric signals. A signal processor receives the detector output signal, the signal processor configured to demodulate the detector output signal by applying a first demodulation signal to a signal responsive to the detector output signal to generate a first demodulator output signal, applying a second demodulation signal to the signal responsive to the detector output signal to generate a second demodulator output signal, and applying a third demodulation signal to the signal responsive to the detector output signal to generate a third demodulator output signal. The signal processor selects the first demodulation signal, the second demodulation signal and the third demodulation signal to reduce crosstalk between the first, second, and third demodulator signals using, at least in part, first and second calibration data. The first calibration data includes calibration data wherein the first signal source, the second signal source, and the third signal source are operated singly, and wherein the second calibration data comprises calibration data wherein the first signal source, the second signal source, and the third signal source are operated in pairs.

One embodiment includes an apparatus for measuring one or more parameters in a subject. The apparatus includes, three or more sources, at least one detector configured to sense electromagnetic radiation produced by the three or more sources, an output of the at least one detector comprising a detector signal, and a signal processor. The signal processor receives the detector output signal, the signal processor configured to demodulate the detector output signal by applying a first demodulation signal to a signal responsive to the detector output signal to generate a first demodulator output signal, applying a second demodulation signal to the signal responsive to the detector output signal to generate a second demodulator output signal, and applying a third demodulation signal to the signal responsive to the detector output signal to generate a third demodulator output signal. The signal processor selects the first demodulation signal, the second demodulation signal and the third demodulation signal to reduce crosstalk between the first, second, and third demodulator signals using, at least in part, calibration data, wherein the calibration data is obtained, at least in part, by activating the three or more sources according to a set of activation patterns, wherein the number of activation patterns is greater than the number of sources in the three or more sources. In one embodiment, the activation patterns include a first set of patterns wherein each of the three or more sources is activated singly, and a second set of patterns wherein each of the three or more sources is deactivated singly. In one embodiment, the activation patterns include patterns wherein each of the three or more sources is activated one at a time. In one embodiment, the activation patterns include patterns wherein each of the three or more sources is de-activated one at a time. In one embodiment the activation patterns include patterns where more than half of the sources are activated and the remaining sources are not activated. In one embodiment the activation patterns include patterns where, at any time, more than half of the sources are not activated and the remaining sources are activated.

One embodiment includes an apparatus for measuring one or more blood constituents in a subject, where the apparatus includes a first signal source which applies a first input signal during a first time interval, a second signal source which applies a second input signal during a second time interval, a third signal source which applies a third input signal during a third time interval, a detector, and a signal processor, The detector detects a first parametric signal responsive to the first input signal passing through a portion of the subject having blood therein and detects a second parametric signal responsive to the second input signal passing through the portion of the subject, and a third parametric signal responsive to the third input signal. The detector generates a detector output signal responsive to the first, second, and third parametric signals. The signal processor which receives the detector output signal, the signal processor demodulating the detector output signal by applying a first demodulation signal to a signal responsive to the detector output signal to generate a first demodulator output signal and applying a second demodulation signal to the signal responsive to the detector output signal to generate a second demodulator output signal, the first demodulation signal determined at least in part from a first dataset wherein each of the first, second, and third sources are activated one at a time and a second dataset wherein each of the first, second, and third sources are deactivated one at a time. In one embodiment, the first dataset includes turning on only one of the first, second, or third signal sources and measuring the crosstalk between one of the parametric signals and the non-corresponding output signals. In one embodiment, at least a portion of the first dataset includes turning off only one of the first, second, or third signal sources and measuring the crosstalk between one of the parametric signals and the non-corresponding output signals.

### BRIEF DESCRIPTION OF THE DRAWINGS

The present invention will be described below in connection with the accompanying figures.

FIG. 1 is a block diagram of a multi-channel processing system that uses feedback from one or more outputs to configure the operation of a signal separator that separates a composite signal into a plurality of output signals.

FIG. 2 is a block diagram of a two-channel signal processing system to determine blood oxygen saturation in a subject, wherein illumination is provided by back-to-back Light-Emitting Diodes (LEDs).

FIG. 3 is a block diagram of a multi-channel signal processing system to determine blood constituents (e.g., oxygen saturation) in a subject, wherein illumination is provided by N diodes or illumination sources.

FIG. 4 is a block diagram of a specific embodiment of the multi-channel processing system of FIG. 1.

### DETAILED DESCRIPTION

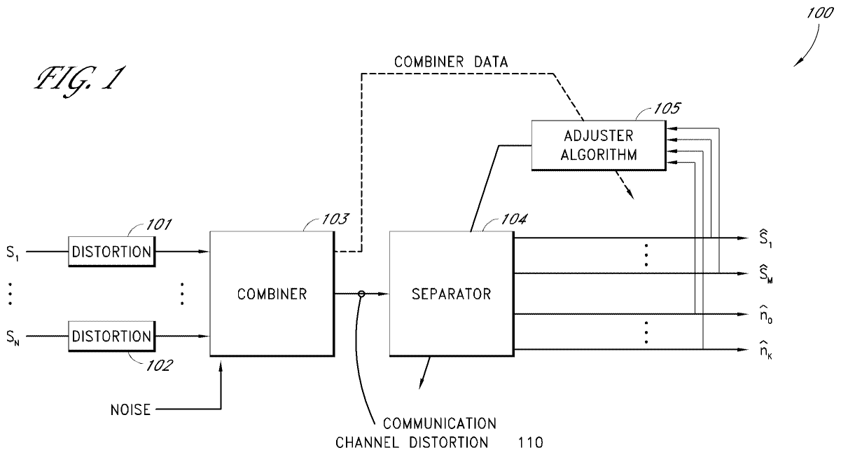

FIG. 1 shows a topology of a multi-channel measurement or communication system 100. The system 100 has a signal combiner 103 for combining one or more input signals S₁ . . . . S_{N} into a composite signal and a signal separator 104 for separating the composite signal into one or more output signals Ŝ₁ . . . .Ŝ_{M} The output signals Ŝ₁ . . . .Ŝ_{M} can include estimates of the input signals S₁ . . . . S_{N}. The input signals S₁ . . . . S_{N} are corrupted by pre-combination distortion 101-102 respectively, and optionally, by combination distortion in the signal combiner 103. The combiner 103 combines the N input signals into a composite signal (or composite signals). The combiner 103 can combine signals by addition, subtraction, multiplication, division, modulation, non-linear processes, linear processes, estimation, combinations thereof, etc. The composite signal is provided through a communication channel to the separator 104. The composite signal is distorted by communication channel distortion 110. The separator 104 separates the composite signal into M output signals, where M can be less than N, greater than N, or equal to N. In one embodiment, the separator also provides one or more additional output signals \hat{n}₀ . . . \hat{n}ₖ corresponding to estimates of other signals, such as, for example, noise signals, error signals, etc.

Due to errors in the system 100, the output signals Ŝ₁ . . . .Ŝ_{M} are typically not exact copies of the input signals, but rather are estimates of the input signals. The accuracy of these estimates is a measure of system performance. The pre-combination distortion 101-102, the combiner distortion, and/or the channel distortion 110 tend to introduce crosstalk between the channels and thereby corrupt the output signals. The pre-combination distortion 101-102, combiner distortion, and the channel distortion 110 can be caused by variations in manufacturing tolerances, delay, movement, temperature effects, degradation of components due to age or other factors, noise, etc.

A module 105 is provided to configure the separator 104 to improve the quality of the separation function and thereby improve the quality of the output signals. One or more of the output signals from the separator are provided to the module 105 to provide feedback regarding the quality of the output signals and/or feedback regarding the operation of the separator 104. The module 105 uses feedback from one or more of the output signals Ŝ₁ . . . .Ŝ_{M} (and, optionally, the output signals \hat{n}₀ . . . \hat{n}ₖ) to monitor the quality of the separation function and to provide control information to control the operation of the separator. In one embodiment, the module 105 is configured by using configuration data obtained from the combiner 103. Such configuration data can be obtained by calibration procedures that test the operation of the combiner 103 before or during system use.

In one embodiment, the module 105 configures demodulators in the signal separator 104 using, at least in part, calibration data obtained during a calibration period. For example, in one embodiment involving a two channel system, the calibration data includes first and second calibration data corresponding to the first and second output signals during a first time period, and third and fourth calibration data corresponding to the first and second demodulated output signals during a second time period. In one embodiment, the second transmitter is turned off during the first time period, and the first transmitter is turned off during the second time period. In one embodiment, the module 105 configures the first demodulation signal and the second demodulation signal by adjusting initial parameters that define the first demodulation signal and the second demodulation signal. The configuration module adjusts the initial parameters using, at least in part, the calibration data obtained during a calibration period.

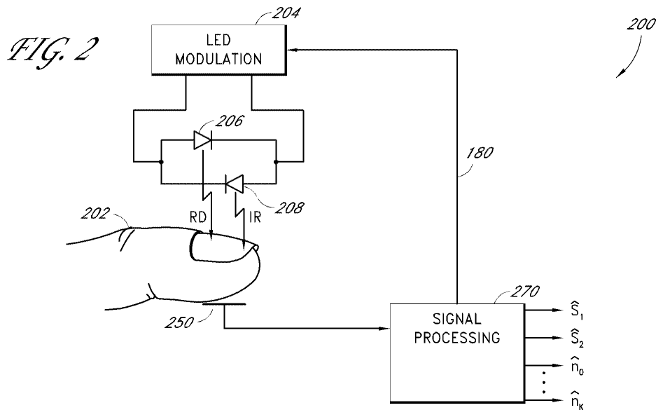

FIG. 2 is a block diagram of a two-channel signal processing system 200 that fits the general topology shown in FIG. 1. The system 200 is configured to determine one or more blood constituents in a subject, such as, for example, a human subject. In the example presented, the measurements are performed on a portion of the subject, such as a finger 202. An LED modulation circuit 204 drives a pair of back-to-back light emitting diodes (LEDs) 206, 208 by applying a periodic signal to the two light emitting diodes 206, 208. Light from the diodes 206, 208 passes through the finger 202 and is detected by a detector 250. An output from the detector 250 is provided to a signal processing block 270. A control output from the signal processing block 270 is provided to the LED modulation circuit 204. The signal processing block 270 also provides outputs Ŝ₁ and Ŝ₂ corresponding to the light detected from the diodes 206, 208, and, optionally, output signals \hat{n}₀ . . . \hat{n}ₖ corresponding to estimates of noise or other signals.

In one embodiment, the LED 206 is selected to emit electromagnetic energy in the red visible light range, and has a wavelength of, for example, approximately 660 nanometers. The LED 208 is selected to emit electromagnetic energy in the infrared range, and has a wavelength of, for example, approximately 905 nanometers. The LED modulation circuit 204 supplies current to activate the LEDs 206 and 208. Each LED is activated for a time period t which can be different for the different LEDs. The pulses from the LEDs 206 and 208 repeat with a periodicity T.

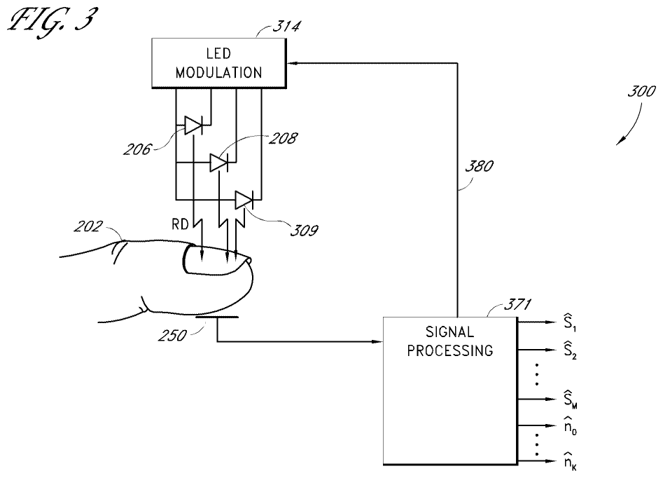

FIG. 3 is a block diagram of a multi-channel signal processing system 300 that also fits the topology shown in FIG. 1. Like the system 200, the system 300 is configured to determine blood oxygen saturation or other blood constituents in a subject, such as, for example, a human subject. In FIG. 3, an LED modulation circuit 314 drives N diodes, where N is two or greater, thus allowing greater flexibility than the system 200. In FIG. 3, the diodes 206 and 208 are shown, along with an N'th diode 309, it being understood that the diode 309 is omitted if N=2. The LED modulation circuit 314 is configured to allow the diodes 206, 208, and 309 to be driven independently such that the diodes can be driven at separate times or in overlapping time periods if desired. Light from the diodes 206, 208, 309 passes through the finger 202 and is detected by the detector 250. The output from the detector 250 is provided to a signal processing block 371. A control output from the signal processing block 371 is provided to the LED modulation circuit 314. The signal processing block 371 also provides outputs S₁ through S_{M}, where M is greater than or equal to one, but need not be equal to N.

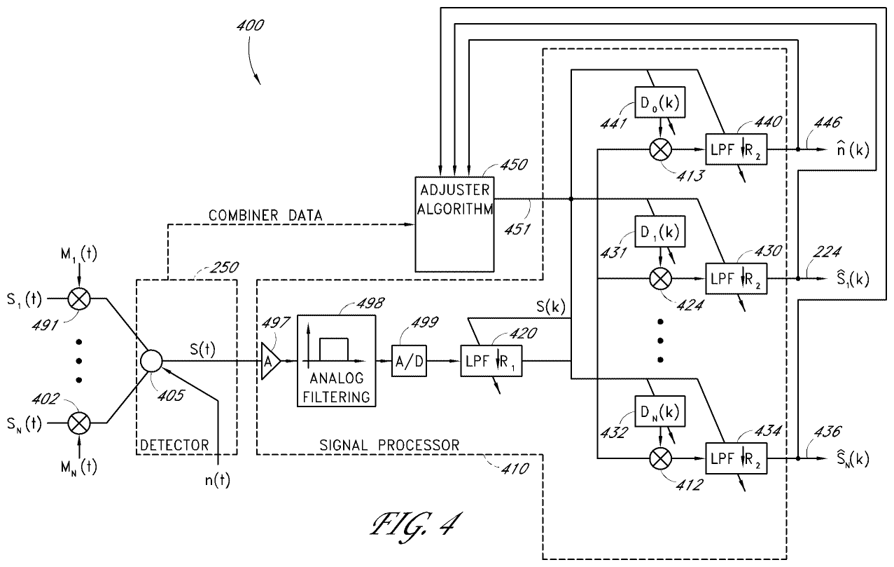

FIG. 4 shows one embodiment of an adjustable multi-channel modulator/demodulator system 400. The system 400 is based on the topology of the system 100 and can be used in a system for measuring blood constituents (e.g., pulse oximetry, carboxyhemoglobin, etc.) as shown in FIGS. 2 and 3. In the system 400, an input S₁(t) and a modulation input M₁(t) are provided to a first modulator 491. A signal input S_{N}(t) and a modulation input MN (t) are provided to an N^{th} modulator 402.

The photodetector 250 is modeled as an adder 405. The outputs of the modulators 491 and 402 are added together in the adder 405, in the presence of noise n(t) to generate a composite signal M(t) where:

$$S ( t ) = {S}_{1} ( t ) {M}_{1} ( t ) + … + {S}_{N} ( t ) {M}_{N} ( t ) + n ( t ) \qquad ( 1 )$$

The S(t) signal output of the adder 405 (i.e., the output of the detector 250) is applied to the input of a signal-processing block 410. Within the signal-processing block 410, the signal S(t) is passed through an amplifier 497 and through an analog bandpass filter 498. The analog bandpass filter 498 provides anti-aliasing and removal of low frequency noise and DC. The desired signal components in the signals Sᵢ(t) are frequency shifted by the operation of the modulation signals Mᵢ(t) and are passed by the analog bandpass filter 498.

The output of the analog bandpass filter 498 is sampled by an analog-to-digital converter 499 and converted therein to digital signals and provided to an input of an optional decimation block 420.

The filtered (and, optionally, decimated) signal S(t) is sampled to produce a sampled-data signal S(k) that is provided to the first input of the first mixer 424, to the first input of the N'th mixer 412, and to the first input of a noise channel mixer 413. A first demodulating signal D₁(k) is provided to a second input of a first mixer 424 from a signal generator 431. The N^{th} demodulating signal D_{N}(k) is provided to an N^{th} mixer 412 from an output of a signal generator 432. The noise demodulating signal D₀(k) is provided to the noise channel mixer 413 from an output of a signal generator 441. A control input to each of the signal generators 431, 432, and 441 is provided by the output of the adjuster algorithm 450. In yet another embodiment, the adjuster algorithm 450 may also be controlled by other signal processing elements downstream of the signal processor 400.

The outputs of the mixers 413, 424, and 412 are provided as respective inputs to decimation blocks 440, 430, and 434 respectively. Each of the decimation blocks 440, 430, and 434 has a control input provided by the output of the adjuster algorithm block 450. The output of the decimation block 440 is an estimate of the signal n(t) and it is provided to an input of the adjuster algorithm block 450. In an alternate embodiment, the signal estimates Ŝ₁ (k) are also provided to the adjuster algorithm block 450.

An output of the decimator 430 is a signal Ŝ1(k), which, as discussed above, is an estimate of the signal S₁(k) (where signal S₁(k) corresponds to a sampled-data representation of signal S₁(t)). Likewise, the output of the decimation block 434 is an estimate of the signal signal S_{N} (k). As shown above, the selection of the demodulating signals Dᵢ(t) for i=0 . . . . N in accordance with the present invention substantially reduces or eliminates the effects of noise in the output signals Ŝ₁(k) and n(k), and also substantially reduces or eliminates crosstalk between the signals.

When the system 400 is used in connection with a blood constituent measurement system as shown in FIGS. 2 and 3, the red LED 206 provides a light intensity represented as I_{RD}, and the infrared LED 208 provides a light intensity represented as I_{IR}. The effects of turning the LEDs 206, 208 on and off on a periodic bases are modeled by the first multiplier or modulator 290 which applies a first modulation signal M₁(t) to the red light intensity to generate a modulated red signal I_{IRMOD}(t) and by a second multiplier the modulator 292 which applies a second modulation signal M₂(t) to the infrared light intensity to generate a modulated infrared signal I_{IRMOD}(t). The modulated light red signal and the modulated infrared signal are applied to the finger 202, or other body portion, as described above. The blood in the finger 202 has a volume and scattering components, which vary throughout each cardiac cycle. The blood carries oxygen and other materials therein. The oxygen content is a function of both the blood volume and the concentration of the oxygen in the blood volume. The concentration of the oxygen in the blood volume is generally measured as blood oxygen saturation for reasons which are described in full in U.S. Pat. Nos. 5,482,036 and 5,490,505, both of which are hereby incorporated by reference in their entirety. As further described in the two referenced patents, the blood oxygen saturation is determined by comparing the relative absorption of the red light and the infrared light in the finger 202. The comparison is complicated by the noise caused by movement, ambient light, light scattering, and other factors. The signals S₁(t) and S₂(t) represent the effect of the time-varying volume and scattering components of the blood in the finger 202 on the red light and the infrared light, respectively, passing through the finger 202 from the LEDs 206, 208 to the detector 250.

As shown in FIG. 4, a set of N+1 signals Sᵢ[k]i=1 . . . . N, and n(k) are sampled at a desired sample rate. The signals are combined according to the formula:

$$S ( k ) = {M}_{1} ( k ) {S}_{1} ( k ) + … + {M}_{N} ( k ) {S}_{N} ( k ) + n ( k ) \qquad ( 2 )$$

In one embodiment, each of the decimators 420, 440, 430, and 434 includes a digital lowpass filter and a sample rate compressor. In one embodiment, the characteristics of the digital lowpass filters (e.g., the number of filter coefficients and values of the filter coefficients) and the sample rate compression factor of each decimator are fixed. In one embodiment, the characteristics of the digital lowpass filters (e.g., the number of filter coefficients or values of the filter coefficients) and the sample rate compression factor of each decimator are provided by the adjustment algorithm 450. The signal generators 431, 432 and 441 generate the demodulation sequences for the demodulators 424, 412, and 413 respectively. The demodulation sequences produced by the signal generators 431, 432 and 441 are controlled by the adjuster algorithm 450.

In one embodiment, the adjuster algorithm 450 adjusts the pre-demodulation decimation rate R₁ (in the demodulator 420), and the post-demodulation decimation rate R₂ (in the demodulators 430, 434 and 440) according to the noise in the noise estimate \hat{n}(k) and (optionally) according to the signals Ŝᵢ(k). The product R₁R₂ is the total decimation rate from the signal S(k) at the output of the A/D converter 499 to the signals Ŝᵢ (k) at the output of the signal processing block 400. The adjuster algorithm may adjust R₁ and R₂ such that the product R₁R₂ varies, or the adjuster algorithm may adjust R₁ and R₂ such that the product R₁R₂ is substantially constant. Typically, the adjuster algorithm will keep the R₁R₂ product constant so that the signal processing blocks downstream of the signal processor 400 will operate at a substantially constant sample rate.

In one embodiment, the adjuster algorithm 450 adjusts the demodulation signals Dᵢ(k) to reduce or eliminate crosstalk. In one embodiment, the adjuster algorithm 450 reduces crosstalk by configuring the demodulators, as discussed in more detail below.

One skilled in the art will recognize that the lowpass filters provided in connection with the decimation blocks can provide other filter functions in addition to lowpass filtering. Thus, for example, the lowpass filters 420, 430, 440, and 450, and the decimators 420, 430, 434, and 440 can provide other filter functions (in addition to lowpass filtering) such as, for example, bandpass filtering, bandstop filtering, etc. Moreover, the post-demodulation decimation rate R₂ need not be the same for each output channel. Thus, for example, in FIG. 4, the decimator 440 can have a first decimation rate R₂=r₁ while the decimators 430 and 434 have a second decimation rate R₂=r₂.

The demodulators above are described in terms of digital signal processing on sampled data. Thus, the demodulator signals are written Dᵢ(k). The demodulators and the filtering associated with the demodulators can be done in using analog processing (using time-domain demodulator signals Dᵢ(t)) or on sampled data signals (using digital-domain demodulator signals Dᵢ(k)). For convenience, the following development describes the demodulator signals primarily in the time domain, with the understanding that the modulators can be implemented using digital signal processing or analog processing.

The characteristics of the demodulation signals D₁(t) and D₂(t) affect how much crosstalk is seen in the output signals. In an diagonal system, that is, when the demodulator has been diagonalized, there is, ideally, no crosstalk. The first output signal Ŝ₁(k) is an estimate (or approximation) to the signal S₁(t). Similarly, the second output signal Ŝ₂ (t) is an estimate (or approximation) to the signal S₂(t). When the composite signal S(t) is a linear combination of the signals S₁(t), then the relationship between the signals Sᵢ(t) and the signals Ŝᵢ(t). When M₁=cos ωt, M₂=sin ωt, n(t)=0, and there is no distortion (e.g., no pre-combination, combiner, or channel distortion) then:

$$S ( t ) = {S}_{1} ( t ) cos ω t + {S}_{2} ( t ) sin ω t \qquad ( 3 )$$

$$Then :$$

$${\hat{S}}_{1} ( t ) = LP [ {D}_{1} ( t ) S ( t ) ] \qquad ( 4 )$$

$${\hat{S}}_{2} ( t ) = LP [ {D}_{2} ( t ) S ( t ) ] \qquad ( 5 )$$

$$If :$$

$${D}_{1} ( t ) = 2 cos ω t \qquad ( 6 )$$

$${D}_{2} ( t ) = 2 sin ω t \qquad ( 7 )$$

$$then$$

$${D}_{1} ( t ) S ( t ) = 2 {S}_{1} ( t ) {cos}^{2} ω t + 2 {S}_{2} ( t ) sin ω t cos ω t = {S}_{1} ( t ) - {S}_{1} ( t ) cos 2 ω t + {S}_{2} ( t ) sin 2 ω t \qquad ( 8 )$$

After lowpass filtering to remove the terms with a frequency of 2 ωt and higher

$${\hat{S}}_{i} ( t ) = {S}_{1} ( t ) \qquad ( 9 )$$

$$Similarly for$$

$${\hat{S}}_{2} ( t ) = P [ {D}_{2} ( t ) S ( t ) ] \qquad ( 10 )$$

$$then$$

$${D}_{2} ( t ) S ( t ) = 2 {S}_{1} ( t ) sin ω t cos ω t + 2 {S}_{1} ( t ) {sin}^{2} ω t = {S}_{2} ( t ) - {S}_{2} ( t ) cos 2 ω t + {S}_{1} ( t ) sin 2 ω t \qquad ( 11 )$$

After lowpass filtering to remove the terms with a frequency of 2 ωt

$${\hat{S}}_{2} ( t ) = {S}_{2} ( t ) \qquad ( 12 )$$

In the above analysis, it was assumed that there are no time delays or phase shifts in the signal S(t), and thus, configuration is relatively straightforward.

When an unknown delay (or phase error) is introduced, then the signals are no longer diagonal. Consider, for example, the situation when a delay A is introduced into the composite signal. Then:

$$S ( t ) = cos ω ( t - Δ ) {S}_{1} ( t - Δ ) + sin ω ( t - Δ ) {S}_{2} ( t - Δ )$$

It then follows that:

$${\hat{S}}_{1} ( t ) = LP [ 2 cos ω t ( cos ω ( t - Δ ) {S}_{1} ( t - Δ ) + sin ω ( t - Δ ) {S}_{2} ( t - Δ ) ) ] = LP [ 2 cos ω t ( cos ω t cos ωΔ + sin ω t sin ωΔ ) {S}_{1} ( t - Δ ) ] + LP [ 2 cos ω t ( sin ω t cos ωΔ + cos ω t sin ωΔ ) {S}_{2} ( t - Δ ) ] = cos ωΔ {S}_{1} ( t - Δ ) - sin ωΔ {S}_{2} ( t - Δ )$$

The above equations can be expressed in matrix form as:

$$[ \begin{matrix}{\hat{S}}_{1} ( t ) \\ {\hat{S}}_{2} ( t )\end{matrix} ] = [ \begin{matrix}cos ωΔ & - sin ωΔ \\ sin ωΔ & cos ωΔ\end{matrix} ] [ \begin{matrix}{S}_{1} ( t - Δ ) \\ {S}_{2} ( t - Δ )\end{matrix} ] \qquad ( 13 )$$

$$Then$$

$$[ \begin{matrix}{S}_{1} ( t - Δ ) \\ {S}_{2} ( t - Δ )\end{matrix} ] = [ \begin{matrix}cos ωΔ & sin ωΔ \\ - sin ωΔ & cos ωΔ\end{matrix} ] [ \begin{matrix}{\hat{S}}_{1} ( t ) \\ {\hat{S}}_{2} ( t )\end{matrix} ] \qquad ( 14 )$$

The above equation can be expressed as

$$[ \begin{matrix}{S}_{1} ( t - Δ ) \\ {S}_{2} ( t - Δ )\end{matrix} ] = [ \begin{matrix}cos ωΔ & sin ωΔ \\ - sin ωΔ & cos ωΔ\end{matrix} ] \cdot LP [ \begin{matrix}{D}_{1} ( t ) S ( t ) \\ {D}_{2} ( t ) S ( t )\end{matrix} ] = LP [ [ \begin{matrix}cos ωΔ & sin ωΔ \\ - sin ωΔ & cos ωΔ\end{matrix} ] [ \begin{matrix}{D}_{1} ( t ) \\ {D}_{2} ( t )\end{matrix} ] S ( t ) ] = LP [ [ \begin{matrix}{\overline{D}}_{1} ( t ) \\ {\overline{D}}_{2} ( t )\end{matrix} ] S ( t ) ] \qquad ( 15 )$$

$$where$$

$${\overline{D}}_{1} ( t ) = cos ωΔ {D}_{1} ( t ) + sin ωΔ {D}_{2} ( t ) = 2 cos ωΔcosω t + 2 sin ωΔsinω t$$

and similarly for D̅₁(t). Thus the modified demodulation functions D̅₁(t) and D̅₂(t) can be expressed as a linear combination of basis functions. If the time delay Δ can be predicted, then the demodulator functions can be calculated and programmed into the communication system. However, in many cases the time delay Δ is not known or changes over time. As described below, the demodulator functions can be determined by system calibration procedures.

When an unknown phase shift (or phase error) is introduced, then there may be crosstalk in the system. Consider, for example, the situation when a phase error φ₁ occurs in the signal S₁(t) and a phase error φ₂ occurs in the signal S₂(t). The phase errors can be caused by intrinsic properties of the components, intrinsic properties of the system, component variations, time delays, etc. In the presence of the phase errors:

$${D}_{1} ( t ) S ( t ) = 2 {A}_{1} {S}_{1} ( t ) sin ( ω t + {ϕ}_{1} ) sin ω t + 2 {A}_{2} {S}_{2} ( t ) cos ( ω t + {ϕ}_{2} ) sin ω t = 2 {A}_{1} {S}_{1} ( t ) sin ω t [ sin ω t cos {ϕ}_{1} + cos ω t sin {ϕ}_{1} ] + 2 {A}_{2} {S}_{2} ( t ) sin ω t [ cos ω t cos {ϕ}_{2} + sin ω t sin {ϕ}_{2} ] = {A}_{1} {S}_{1} ( t ) cos {ϕ}_{1} - {A}_{1} {S}_{1} ( t ) cos {ϕ}_{1} cos 2 ω t + {A}_{1} {S}_{1} ( t ) sin {ϕ}_{1} sin 2 ω t + {A}_{2} {S}_{2} ( t ) cos {ϕ}_{2} sin 2 ω t - {A}_{2} {S}_{2} ( t ) sin {ϕ}_{2} + {A}_{2} {S}_{2} ( t ) sin {ϕ}_{2} cos 2 ω t \qquad ( 16 )$$

Thus, after lowpass filtering

$${\hat{S}}_{1} ( t ) = {A}_{1} {S}_{1} ( t ) cos {ϕ}_{1} - {A}_{2} {S}_{2} ( t ) sin {ϕ}_{2} \qquad ( 17 )$$

The above equation shows crosstalk because Ŝ₁(t) depends in part on components of S₂(t) when A₂≠0 and φ₂≠nπ where n=0,±1, ±2 . . .

Similarly,

$${\hat{S}}_{2} ( t ) = {A}_{1} {S}_{1} ( t ) sin {ϕ}_{1} + {A}_{2} {S}_{2} ( t ) cos {ϕ}_{2} \qquad ( 18 )$$

The above equations can be expressed in matrix form as:

$$[ \begin{matrix}{\hat{S}}_{1} ( t ) \\ {\hat{S}}_{2} ( t )\end{matrix} ] = [ \begin{matrix}{A}_{1} cos {ϕ}_{1} - {A}_{2} sin {ϕ}_{2} \\ {A}_{1} sin {ϕ}_{1} - {A}_{2} cos {ϕ}_{2}\end{matrix} ] [ \begin{matrix}{S}_{1} ( t ) \\ {S}_{2} ( t )\end{matrix} ] \qquad ( 19 )$$

$$After inversion$$

$$[ \begin{matrix}{S}_{1} ( t ) \\ {S}_{2} ( t )\end{matrix} ] = \frac{1}{cos ( {ϕ}_{1} - {ϕ}_{2} )} [ \begin{matrix}\frac{cos {ϕ}_{2}}{{A}_{1}} & \frac{sin {ϕ}_{1}}{{A}_{1}} \\ - \frac{sin {ϕ}_{2}}{{A}_{2}} & \frac{cos {ϕ}_{1}}{{A}_{2}}\end{matrix} ] [ \begin{matrix}{\hat{S}}_{1} ( t ) \\ {\hat{S}}_{2} ( t )\end{matrix} ] \qquad ( 20 )$$

$$[ \begin{matrix}{S}_{1} ( t ) \\ {S}_{2} ( t )\end{matrix} ] = \frac{1}{cos ( {ϕ}_{1} - {ϕ}_{2} )} [ \begin{matrix}\frac{cos {ϕ}_{2}}{{A}_{1}} & \frac{sin {ϕ}_{1}}{{A}_{1}} \\ - \frac{sin {ϕ}_{2}}{{A}_{2}} & \frac{cos {ϕ}_{1}}{{A}_{2}}\end{matrix} ] [ \begin{matrix}{D}_{1} ( t ) \\ {D}_{2} ( t )\end{matrix} ] S ( t ) \qquad ( 21 )$$

$$Then$$

$$[ \begin{matrix}{S}_{1} ( t ) \\ {S}_{2} ( t )\end{matrix} ] = [ \begin{matrix}{\overline{D}}_{1} ( t ) \\ {\overline{D}}_{2} ( t )\end{matrix} ] S ( t ) \qquad ( 22 )$$

Where D̅₁(t) are modified demodulation functions, given by:

$$[ \begin{matrix}{\overline{D}}_{1} ( t ) \\ {\overline{D}}_{2} ( t )\end{matrix} ] = \frac{1}{cos ( {ϕ}_{1} - {ϕ}_{2} )} [ \begin{matrix}\frac{cos {ϕ}_{2} sin ω t + sin {ϕ}_{1} cos ω t}{{A}_{1}} \\ \frac{- sin {ϕ}_{2} sin ω t + sin {ϕ}_{1} cos ω}{{A}_{2}}\end{matrix} ] \qquad ( 23 )$$

The modified demodulation functions have the form

$${\overline{D}}_{1} ( t ) = {\sum }_{j = 1}^{N} {α}_{j} {Φ}_{j} ( t ) \qquad ( 24 )$$

Choosing the coefficients αⱼ to eliminate crosstalk configures the modulator.

In one embodiment, the coefficients αⱼ can be fixed coefficients computed using known properties of the system. However, as system properties change over time, such fixed coefficients may lead to unacceptably high levels of crosstalk. Moreover, variations from device to device may cause the fixed coefficients to give unacceptably high levels of crosstalk.

Higher performance (that is, lower crosstalk) can be obtained by computing the coefficients as part of a system calibration or initialization procedure. Such calibration can be performed during system startup (e.g., when the system is turned on, or when the system begins processing data, etc.) and/or at regular intervals. In one embodiment, the adjuster algorithm 450 computes the coefficients αⱼ from the calibration data. In one embodiment, the coefficients αⱼ are chosen by making four calibration-type measurements to measure four parameters ξ₁₁, μ₁₂, ξ₁₃, and ξ₂₂, where:

$${ξ}_{11} = {\hat{S}}_{1} {\vert}_{{S}_{1} = A , {S}_{2} = 0} = {Aa}_{1} cos {ϕ}_{1} \qquad ( 25 )$$

$${ξ}_{12} = {\hat{S}}_{1} {\vert}_{{S}_{1} = 0 , {S}_{2} = B} = {Ba}_{2} sin {ϕ}_{2} \qquad ( 26 )$$

$${ξ}_{21} = {\hat{S}}_{2} {\vert}_{{S}_{1} = A , {S}_{2} = 0} = {Aa}_{1} sin {ϕ}_{1} \qquad ( 27 )$$

$${ξ}_{22} = {\hat{S}}_{2} {\vert}_{{S}_{1} = 0 , {S}_{2} = B} = {Ba}_{2} cos {ϕ}_{2} \qquad ( 28 )$$

where A and B are amplitudes. Then

$${a}_{1} = \frac{\sqrt{{ξ}_{11}^{2} + {ξ}_{21}^{2}}}{A} \qquad ( 29 )$$

$${a}_{2} = \frac{\sqrt{{ξ}_{12}^{2} + {ξ}_{22}^{2}}}{B} \qquad ( 30 )$$

$$tan {ϕ}_{1} = \frac{{ξ}_{21}}{{ξ}_{11}} \qquad ( 31 )$$

$$tan {ϕ}_{2} = - \frac{{ξ}_{12}}{{ξ}_{22}} \qquad ( 32 )$$

From the above equations, it is evident that crosstalk depends only on φ₁ and φ₂. Moreover, φ₁ and φ₂ can be chosen to eliminate crosstalk without knowing A or B. This is useful for systems such as pulse oximetry systems where absolute measurements of a channel are difficult or impractical, but where relative measurements (e.g., channel-to-channel measurements) are practical.

The demodulation signals D₁(t) can be generated using the values of φ₁ and φ₂ from the above equations. Alternatively, the demodulation signals D₁(t) can be generated from quadrature components as:

$${D}_{1} ( t ) = {b}_{11} sin ω t + {b}_{12} cos ω t \qquad ( 33 )$$

$${D}_{2} ( t ) = {b}_{21} sin ω t + {b}_{22} cos ω t \qquad ( 34 )$$

where the coefficients bᵢⱼ are computed from φ₁ and φ₂.

In one embodiment, the demodulation functions are adapted from baseline coefficients, which are then improved through a calibration or initialization procedure to produce actual coefficients. The baseline coefficients are typically obtained from known properties of the system. The actual coefficients are usually relatively close in value to the baseline coefficients. This provides one way to assess the operational status of the system and to evaluate the calibration procedure. In one embodiment, if the actual coefficients are too different from the baseline parameters then it is assumed that the calibration procedure failed in some manner or that the equipment has failed in some manner, and appropriate measures can be taken (e.g., alert the operator, sound a warning, etc.)

To find the actual coefficients, the demodulation functions are initially given by:

$${D}_{1} ( t ) = {α}_{11} sin ω t + {α}_{12} cos ω t \qquad ( 35 )$$

$${D}_{2} ( t ) = {α}_{21} sin ω t + {α}_{22} cos ω t \qquad ( 36 )$$

Where the coefficients αᵢⱼ are the baseline coefficients determined from known or assumed properties of the signal S(t). For example, in one embodiment αᵢⱼ=δᵢⱼ. In one embodiment, where initial estimates are available for φ₁ and φ₂, then the values of αᵢⱼ can be computed as discussed above.

The crosstalk reduction obtained using demodulation functions based on the coefficients αᵢⱼ can often be improved by computing new coefficients αᵢⱼ and corresponding new demodulation functions D̅ᵢ(t) where:

$${\overline{D}}_{1} ( t ) = {\overline{α}}_{11} sin ω t + {\overline{α}}_{12} cos ω t \qquad ( 37 )$$

$${\overline{D}}_{2} ( t ) = {\overline{α}}_{21} sin ω t + {\overline{α}}_{22} cos ω t \qquad ( 38 )$$

The process of finding the coefficients α̅ᵢⱼ begins by measuring two data sets, x₁(t) and x₂(t), as follows:

$${x}_{1} ( t ) = S ( t ) {\vert}_{{S}_{1} = A , {S}_{2} = 0} \qquad ( 39 )$$

$${x}_{2} ( t ) = S ( t ) {\vert}_{{S}_{1} = A , {S}_{2} = B} \qquad ( 40 )$$

The data sets x₁(t) and x₂(t) are used to enforce the following constraint:

$${\int }_{0}^{n T} {x}_{1} ( t ) {\overline{D}}_{i} ( t ) d = 0 \qquad ( 41 )$$

where i=1, 2, n=1, 2, 3 . . . , and T is a time period corresponding to one complete modulation cycle. From the above constraint and the definitions of the demodulation functions, it follows that:

$${\overline{α}}_{11} {\int }_{0}^{n T} {x}_{1} ( t ) sin ω t d t + {\overline{α}}_{12} {\int }_{0}^{n T} {x}_{1} ( t ) cos ω t d t = 0 \qquad ( 42 )$$

$${\overline{α}}_{21} {\int }_{0}^{n T} {x}_{2} ( t ) sin ω t d t + {\overline{α}}_{22} {\int }_{0}^{n T} {x}_{2} ( t ) cos ω t d t = 0 \qquad ( 43 )$$

$$It is convenient to define {γ}_{11} {\int }_{0}^{n T} {x}_{1} ( t ) sin ω t d t \qquad ( 44 )$$

$${γ}_{12} {\int }_{0}^{n T} {x}_{1} ( t ) cos ω t d t \qquad ( 45 )$$

$${γ}_{21} {\int }_{0}^{n T} {x}_{2} ( t ) sin ω t d t \qquad ( 46 )$$

$${γ}_{22} {\int }_{0}^{n T} {x}_{2} ( t ) cos ω t d t \qquad ( 47 )$$

$$and to define {β}_{i j} = \frac{{γ}_{i j}}{{[ {\sum }_{k} {γ}_{i k}^{2} ]}^{1 / 2}} \qquad ( 48 )$$

$$where {\sum }_{k} {β}_{i k}^{2} = 1 \qquad ( 49 )$$

$$Then {\overline{α}}_{11} {β}_{11} + {\overline{α}}_{12} {β}_{12} = 0 \qquad ( 50 )$$

$${\overline{α}}_{21} {β}_{21} + {\overline{α}}_{22} {β}_{22} = 0 \qquad ( 51 )$$

In one embodiment, to reduce crosstalk, it is desired to find the coefficients α̅ₐⱼ closest (in the sense of minimizing some specified error, such as, for example, a least squared error) to the coefficients αᵢⱼ such that the above constraints are satisfied. One solution, obtained by minimizing the least squared error is:

$${\overline{α}}_{i j} = {α}_{i j} - ( {\sum }_{k} {α}_{i k} {β}_{i k} ) {β}_{i j} \qquad ( 52 )$$

The term in parentheses can be described as the baseline crosstalk.

One of ordinary skill in the art will recognize that optimization methods other than least squares can be used. The solution methods for configuration are, for simplicity, described above in terms of a two-channel system. Using the above teachings, the extension to multi-channel systems is straightforward.

In one embodiment, systems with more than two channels can be calibrated using data from multiple calibration data sets. The calibration data sets can use different combinations of sources and/or different activation patterns of sources to provide additional information for calibration. Thus for example, in one embodiment, calibration measurements are obtained by activating the sources one at a time (with the other sources off) and calibration measurements are also obtained by deactivating one source at a time (with the other sources on). Both sets of calibrations measurements can then be used (in whole or in part) to calculate demodulation information.

In one such procedure for a system with 8 sources, data sets x₁-x₁₆ are obtained using the following activation patterns as follows:

$${x}_{1} = LED 1 on ( others off )$$

$${x}_{2} = LED 2 on ( others off )$$

$$⋮$$

$${x}_{8} = LED 8 on ( others off )$$

$${x}_{9} = LED 1 off ( others on )$$

$${x}_{10} = LED 2 off ( others on )$$

$$⋮$$

$${x}_{16} = LED 8 off ( others on )$$

Then using the functions:

$${ϕ}_{2 j , k} = sin ( 2 π {f}_{j} \frac{k}{T} + H {W}_{j} )$$

$${ϕ}_{2 j + 1 , k} = cos ( 2 π {f}_{j} \frac{k}{T} + H {W}_{j} )$$

where HWⱼ are hardware coefficients (such as, for example, known delays in the system), and fⱼ are the modulation frequencies, then the δ matrix can be computed as

$${ζ}_{i , j} = {\sum }_{k = 1}^{♯ p oints} {x}_{i , k} {ϕ}_{j , k}$$

$$ζ = {\underbrace{[ \begin{matrix}{ζ}_{1 , 1} & ⋯ & ⋯ & {ζ}_{1 , 8} \\ ⋮ & & & ⋮ \\ {ζ}_{8 , 1} & & & {ζ}_{8 , 8} \\ ⋮ & & & ⋮ \\ {ζ}_{16 , 1} & ⋯ & ⋯ & {ζ}_{16 , 8}\end{matrix} ]}}_{2 order} \} number of channels$$

Then, under the assumption that the number of channels is equal to 2 time the order, to compute the demodulation coefficients for channel j′ the Λ matrix can be computed as:

$$⋀ = {\underbrace{[ \begin{matrix}{ζ}_{1 , 1} & ⋯ & {ζ}_{1 , 8} \\ ⋮ & & ⋮ \\ {ζ}_{8 , 1} & ⋯ & {ζ}_{8 , 8} \\ {ζ}_{8 , {j}^{′} , 1} & ⋯ & {ζ}_{8 , {j}^{′} , 8}\end{matrix} ] \}}}_{2 \cdot order} 2 order + 1$$

The QR decomposition of A returns Q and R matrices where:

$$Q is ( 2 \times order + 1 ) \times ( 2 \times order + 1 )$$

$$R is ( 2 \times order + 1 ) \times ( 2 \times order )$$

$$Define j = 1 … 8 = 2 \times order$$

$$k = 1 … 8 = 2 \times order$$

$${A}_{j , k} = {R}_{j , k} {b}_{k} = {Q}_{{j}^{′} , k} ← {j}^{′} th row but only 2 \times order values$$

Then calculate \vec{β} where

$${β}_{2 \cdot order} = \frac{{β}_{2 \cdot order}}{{A}_{2 \cdot order , 2 \cdot order}}$$

$$and for i = 2 \cdot order = 1 , … 1$$

$${β}_{i} = \frac{( {b}_{1} {\sum }_{j = i + 1}^{2 \cdot order} {A}_{i , j} {β}_{j} )}{{A}_{i , i}}$$

$$After normalizing \vec{β} by \hat{β} = \frac{\vec{β}}{\vert \vec{β} \vert}$$

Then the new demodulation coefficient vectors are given by:

$${\hat{α}}_{j} = ( {\vec{α}}_{j} \cdot \hat{β} ) {\hat{β}}_{j}$$

where \vec{α}ⱼ are the baseline and/or previous demodulation coefficient vectors.

To test crosstalk for the j^{th} channel, compute

$${ξ}_{k} = {\sum }_{ℓ = 1}^{2 \cdot order} {ζ}_{k , ℓ} {\hat{α}}_{ℓ} ( where \hat{α} corresponds to the {j}^{th} channel$$

$$if k \leq 2 \cdot order then :$$

$$when the {j}^{th} channel is on : {crosstalk}_{k \neq j} = 20 log ( \frac{{ξ}_{k}}{{ξ}_{j}} )$$

$$when the {j}^{th} is channel off {crosstalk}_{k \neq j} 2 \cdot order = 20 log ( \frac{{ξ}_{k} + 2 \cdot order}{{ξ}_{j} + 2 \cdot order} )$$

The example above uses 8 sources by way of example and not by way of limitation. One of ordinary skill in the art will further recognize that other combinations and activation patterns can be used as well. For example, in one embodiment calibration measurements are obtained using activation patterns sources (LEDs) when less than half of the sources are activated, and calibration measurements are obtained using combinations when more than half of the sources are activated. With N sources, up to 2^{N} different combinations of activation patterns are possible using the N sources. Thus, one of ordinary skill in the art will recognize that in various embodiments, the demodulation coefficients can be calculated using calibration data from many different activation patterns of activated and deactivated sources. Further, in the above example, the number of channels was equal to 2 times the order. One of ordinary skill in the art will recognize that the above analysis can also be used whenever number of channels is less than or equal to 2×order.

Although described above in connection with a particular embodiment of the present invention, it should be understood the description of the embodiment is illustrative of the invention and are not intended to be limiting. Although described above in connection with a pulse oximetry system wherein a parameter to be measured is the attenuation of red and infrared light passing through a portion of a subject's body, it should be understood that the method and apparatus described herein can also be used for other measurements where two or more signals are passed through a system to be analyzed. In particular, the present invention can be used to demodulate two combined parametric signals responsive to the system to be analyzed where the two parametric signals have a predetermined timing relationship between them, as described herein. The invention can be used in connection with various physiological parameter measurement systems, such as, for example, systems that measure blood constituents, blood oxygen carboxyhemoglobin, methemoglobin, glucose, etc. Various modifications and applications may occur to those skilled in the art without departing from the true spirit and scope of the invention as defined in the appended claims.

1.-12. (canceled)

13. A method of reducing crosstalk between two signals generated by applying a first pulse and a second pulse to measure a parameter, wherein the first pulse and the second pulse are applied periodically at a repetition rate defining a period, the first pulse being generated during a first interval in each period and the second pulse being generated during a second interval in each period, wherein the first and second pulses are configured to produce first and second parametric signals responsive to the parameter, the first and second parametric signals being received by a detector configured to output a composite signal responsive to the first and second parametric signals, the method comprising:
applying a first demodulation signal to the composite signal to generate a first demodulated output signal, the first demodulation signal including at least one component having at least a first amplitude and a first phase;
applying a second demodulation signal to the composite signal to generate a second demodulated output signal, the second demodulation signal including at least one component having at least a second amplitude and a second phase;
lowpass filtering the first demodulated output signal to generate a first recovered output signal responsive to the first parametric signal;
lowpass filtering the second demodulated output signal to generate a second recovered output signal responsive to the second parametric signal; and
selecting at least one of the first phase, the first amplitude, the second phase, and the second amplitude to reduce crosstalk components in the first recovered output signal and the second recovered output signal.

14. The method of claim 13, further comprising selecting the first phase and/or the second phase to reduce crosstalk components in the first recovered output signal and the second recovered output signal.

15. The method of claim 13, wherein the second interval is spaced apart from the first interval.

16. The method of claim 13, wherein the second interval overlaps at least a portion of the first interval.

17. The method of claim 13, wherein the first phase and the second phase are chosen by applying a first light pulse activation pattern during a first time period and measuring the first recovered output signal during the first time period as a first calibration output, and measuring the second recovered output signal during the first time period as a second calibration output.

18. The method of claim 17, wherein the first phase is computed from a ratio of the first calibration output and the second calibration output.

19. The method of claim 17, further comprising applying a second light pulse activation pattern during a second time period and measuring the first recovered output signal during the first time period as a third calibration output and measuring the second recovered output signal during the second time period as a fourth calibration output.

20. The method of claim 19, further comprising computing the first phase and the second phase from at least the first calibration output, the second calibration output, the third calibration output, and the fourth calibration output.

21. The method of claim 13, wherein the first demodulation signal includes a sum of a first demodulation component having a first amplitude and a second demodulation component having a second amplitude.

22. The method of claim 21, wherein the second demodulation component is in quadrature with the first demodulation component.

23. The method of claim 22, wherein selecting the first phase includes selecting the first amplitude and the second amplitude.

24. The method of claim 22, wherein the first demodulation component and the second demodulation component are sinusoidal and cosinusoidal.

25. The method of claim 13, wherein the first demodulation signal includes a sum of a sinusoidal component having a first amplitude and a cosinusoidal component having a second amplitude.

26. The method of claim 25, wherein the first amplitude and the second amplitude are chosen by a least squares minimization of an error corresponding to the crosstalk.

27. The method of claim 26, wherein the error is integrated over a time period corresponding to an integer number of cycles of the sinusoidal component.

28. The method of claim 13, wherein the composite signal has first and second coefficients to generate the first demodulated output signal.

29. The method of claim 13, wherein the first demodulation signal includes a first component having a first amplitude and a second component having a second amplitude, the first and second components being in quadrature.

30. The method of claim 29, wherein the second amplitude has a predetermined relationship to the first amplitude.

31. The method of claim 30, wherein the predetermined relationship is selected to cause the first demodulated signal to have lower frequency components that include a primary component corresponding primarily to the first component and a residual component corresponding to the second component.

32. The method of claim 31, wherein at least one of the first amplitude and the second amplitude are adjusted to reduce the residual component with respect to the primary component.
